(19) United States
(12) Patent Application Publication
(10) Pub. No.: US 2004/0147988 A1
(43) Pub. Date: Jul. 29, 2004

(54) Method of treatment using one or more of a group of the "THERMA-KLOTH" products

(76) Inventor: Willard M. Stephens, Douglasville, GA (US)

Correspondence Address: Willard M. Stephens, 2376 Fairburn Road, Douglasville, GA, 30135, US

(21) Appl. No.: 10/354,224

(22) Filed: Jan. 29, 2003

(51) Int. Cl. A61F 7/00

(52) U.S. Cl. 607/108

(57) ABSTRACT

A method of treatment for illnesses of, but not limited to humans, related to the common sore throat related to the common cold, flu, hay fever, asthma, allergies, sinus infections, coughing and laryngitis, by the use of, and the inclusion of the THERMA-KLOTH products.

### FIELD OF THE INVENTION

This invention relates directly to human health. More specifically, this invention relates to the treatment and healing of illness conditions associated and related to the common cold.

### BACKGROUND OF THE INVENTION

For centuries there have been illness conditions of humans related to the common cold, flu, hay fever, asthma, allergies, sinus infections, coughing and laryngitis. All of these illnesses have at least on thing in common. That is mucus fluid discharge (a part of the natural body protection system).

Although this mucus discharge is a natural body protector, it can sometimes get out of control. This uncontrolled discharge can, in many cases, cause the common sore throat and other related illnesses. Certain surrounding conditions can also contribute to the causes of the common sore throat. These are dry air, cold air drafts, rapid body cooling as a result of wet clothing against the body, sudden body temperature rise, air laden foreign substances, and breathing cold air.

Further, other illnesses such as strep throat, head congestion, headaches, and chest congestion can stem from a build-up of the uncontrolled mucus fluid discharge. Even further, these illnesses affect other body organs and/or systems causing disruption of normal body functions, and sometimes causing other illnesses. Any of these illnesses can become severe, and in some cases can even cause death.

Concluded from this, the need for a safe method of treating to heal the common sore throat is necessary for good health of humans.

There are hundreds of drug-type medicines sold today to treat illnesses previously mentioned. Most of these medicines have uncomfortable, or possible unhealthy side effects which sometimes can become worse that the original illnesses they were intended to treat.

None of these medicines have yet to cure those illnesses. While giving some relief, they tend only to slow down the symptoms until the body resistances become strong enough to naturally ward off the illness. This sometimes can take weeks or may not help at all.

The use of the THERMA-KLOTH creates a reasonably fast, safe and effective method of treatment for a wide range of the symptoms, afore mentioned, by capturing and containing body heat, and directing it back to the affected body area or areas.

It is this method of treating these illnesses with the use of the THERMA-KLOTH, and the development and inclusion of the THERMA-KLOTH, that I have invented, and submit to be patented as my own invention.

### SUMMARY OF THE INVENTION

The invention is THE METHOD OF TREATMENT resulting from, and directly related to the use of and the inclusion of the THERMA-KLOTH to accomplish the treatment for the common sore throat, and/or related illnesses.

When the THERMA-KLOTH is snugly placed and tucked in a position covering the throat area, or other areas, it captures and contains body heat. This heat in turn radiates back to the body (the throat area, or other areas), which is covered by the THERMA-KLOTH.

This method of treatment using this heat, stimulates blood and lymph circulation. It also aids in the dissolving and breaking up congestive type mucus. This process enhances the recovery time related to throat and/or related illnesses.

Further, this method of treatment can be used effectively in treating some body injuries.

The THERMA-KLOTH products are comprised of multi-layers, more or less, of thermal cloth material, which may vary in composition, arranged and assembled to conform to a comfortable shape, and which may vary in shape and size.

### BRIEF DISCRIPTION OF THE THERMA-KLOTH DRAWING

FIG. 1 and FIG. 2 are basic drawings showing the face and side view typical to, but not limited to, the shape of the THERMA-KLOTH.

### DETAILED DESCRIPTION OF THE THERMA-KLOTH

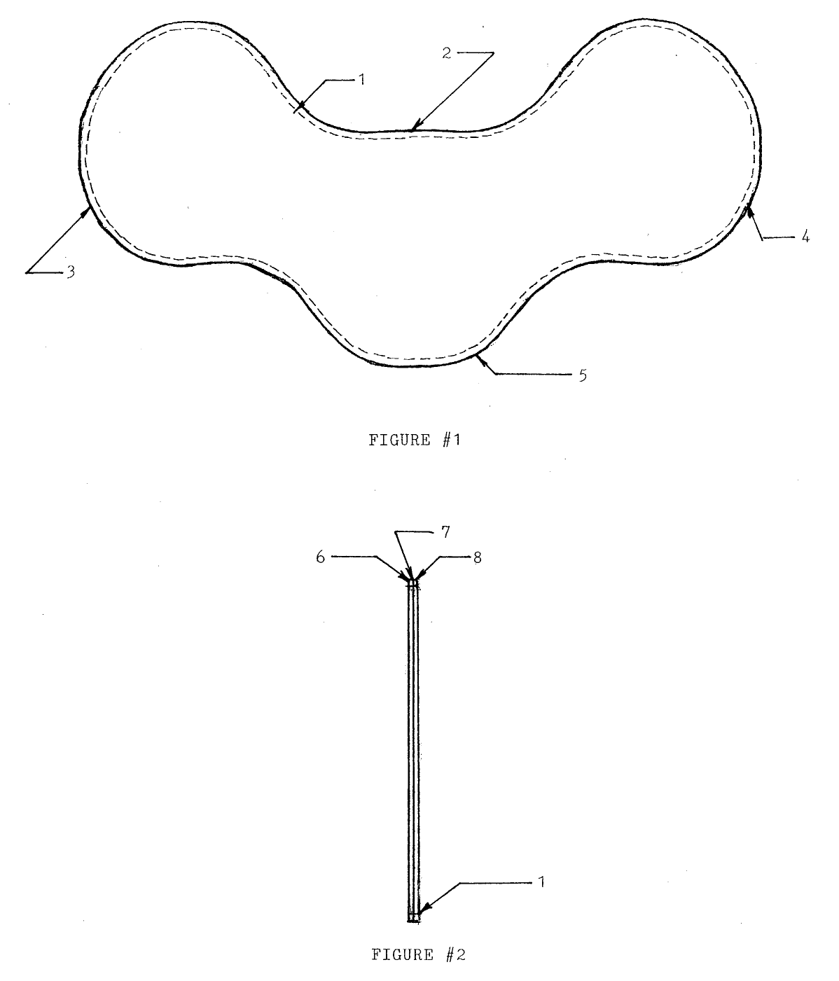

The face view can be either the front or the back. FIG. 1 shows the typical shape of the THERMA-KLOTH. The outer curved shapes are the chin curve 2, the left side tuck 3, the right side tuck 4, and the chest cover flap 5, the assembly stitching 1 reinforces the outer edge, and holds the materials firm. The side view FIG. 2 shows the assembly of the thermal cloth material layers 6, 7 and 8, and their relationship to each other, along with assembly stitching 1.

The THERMA-KLOTH products can vary in composition, shapes, colors, configuration, and insulating ability.

What I claim is:

1. A method of treating to heal illness conditions of, but not limited to, humans, related to the common sore throat related to the common cold, flu, hay fever, asthma, allergies, sinus infections, coughing, and laryngitis by the use of, and the inclusion of the THERMA-KLOTH products, and the development of the THERMA-KLOTH.

2. Assembly of the THERMA-KLOTH products varies in thickness, configuration, color, and shape.

3. Materials comprised in the assembly of the THERMA-KLOTH products vary in their thermal insulating ability.

4. Proper use, afore mentioned, of the THERMA-KLOTH products are used to treat other parts of the body.

5. Using the proper method of treatment, the THERMA-KLOTH products will cure a sore throat.

6. Using the proper method of treatment, the THERMA-KLOTH products will aid in dissolving and breaking up phlegm type of mucus in the head, throat, and chest areas.

7. Using the proper method of treatment, the THERMA-KLOTH products is a method of preventing illness conditions related to the common cold.

8. A method of treatment using the THERMA-KLOTH products are used to treat other parts of the body.

9. That no side-effects occur using the method of treatment to heal and/or the THERMA-KLOTH products afore mentioned.

10. That the same method of treatment can be used to treat living creatures other than humans, such as animals (domestic and/or wild).
